(19) United States
(12) Patent Application Publication
(10) Pub. No.: US 2011/0044927 A1
(43) Pub. Date: Feb. 24, 2011

(54) ODOR ABATEMENT OF ORGANIC WASTE

(75) Inventor: Richard Weschler, Brooklyn, NY (US)

Correspondence Address: STROOCK & STROOCK & LAVAN LLP, 180 MAIDEN LANE, NEW YORK, NY, 10038, US

(73) Assignee: ACETO CORPORATION, Late Sucess, NY (US)

(21) Appl. No.: 11/661,514

(22) PCT Filed: Aug. 26, 2005
(86) PCT No.: PCT/US05/31183

§ 371 Date: Jun. 12, 2008

Related U.S. Application Data
(60) Provisional application No. 60/605,277, filed on Aug. 26, 2004.

(51) Int. Cl. A61L 11/00

(52) U.S. Cl. 424/76.6

(57) ABSTRACT

The present invention relates to a system and a method of abating hydrogen sulfide gas emitted by or generated by organic matter. Certain embodiments of the present invention relate to contacting hydrogen sulfide gas with metals such as copper, iron, zinc, silver or mixtures thereof, and other components. Other embodiments of the present invention relate to contacting hydrogen sulfide gas with a solution of metals such as copper, iron, zinc or mixtures thereof, and other components.

### CROSS REFERENCE TO RELATED APPLICATION

This application claims the benefit of Provisional Application No. 60/605,277, filed on Aug. 26, 2004, the contents of which are incorporated herein by reference. This application is also a continue-in-part application of U.S. Ser. No. 10/799,434, filed Mar. 11, 2004, which itself claimed the benefit of provisional application 60/512,327, filed on Oct. 17, 2003. The contents of both applications are incorporated herein by reference.

### BACKGROUND OF INVENTION

The invention relates generally to the abatement of toxic and/or noxious gases, and more particularly to the abatement of hydrogen sulfide gas emitted or generated by organic waste sources.

Organic waste is produced in huge quantities in operations such as large scale animal breeding farms and wastewater treatment facilities that treat residential sewage. For surrounding areas, the bad odor associated with gases, such as amines, ammonia and hydrogen sulfide, emitted from these operations due to the biochemical decomposition of organic wastes, are considered a public nuisance and a health hazard.

Animal breeding farms raise animals in large numbers for food consumption by humans or for other useful purposes. These large concentrations of animals lead to the production of large amounts of solid and liquid waste materials. In New York State alone, there are over 10,000 dairy farms with over 1,000,000 cows, each cow producing at least 80 lbs of manure per day. Typically, the waste material at these farms is collected and maintained in waste holding pits, such as lagoons, situated beneath or adjacent to a facility in which the animals are housed. Any waste residue left behind is removed by washing with water. The water is then collected and the water and manure are sent to a lagoon. In the lagoon, the manure is concentrated by separating out the liquid phase and then treated. Sometimes, the wastes are stored in waste holding tanks, waste wetlands or other types of waste holding facilities.

The waste material is commonly allowed to degrade in pits by the activity of naturally occurring microorganisms. It is also common to add compositions including specific microorganisms. As the pits become full, the waste materials are removed usually by pumping and the degraded waste materials are then often spread across crop fields for their fertilizer value.

Common fertilizers include ammonia, ammonium nitrate and urea. Green plants are able to utilize several chemical forms of nitrogen, especially NH₃ and NO⁻₃. Plants use nitrogen to synthesize several nitrogen containing compounds including proteins. Manure is rich in these forms of nitrogen that also emit undesirable hydrogen sulfide. Therefore, there exists a need to treat the manure to eliminate hydrogen sulfide odors, increasing its desirability as a fertilizer.

At wastewater treatment facilities, a large flow of raw sewage containing considerable amounts of organic waste is handled on a daily basis. The sewage undergoes some anaerobic digestion causing it to emit gases, such as hydrogen sulfide gas, into the air. In addition to being unpleasant to smell, hydrogen sulfide is also toxic to sewer workers and can corrode expensive treatment equipment. In the gaseous phase, hydrogen sulfide will be adsorbed by moisture on the exposed areas of pipes to form thin layers of acid. In these layers relatively high concentration levels of acid will corrode the pipe material leading eventually to line failures. Such corrosion can shorten the life of any exposed metal and concrete equipment and fixtures. Because of the hydrogen sulfide's highly offensive odors, health hazards and toxic effects, there exists a need to treat either the sewage and the air or both to reduce or neutralize the effects of the hydrogen sulfide gas.

Accordingly, there is a need for improved system and method for the abatement of organic waste.

### SUMMARY OF INVENTION

Generally speaking, in accordance with the invention, a system and method for reducing air, land and water pollutants is provide, which includes treating hydrogen sulfide emitted from organic waste is provided. Sources of the hydrogen sulfide gas include agricultural waste from feedlots and the like and municipal waste from sewage systems and the like.

Certain embodiments of the invention relate to contacting hydrogen sulfide gas with metals, such as copper, iron, zinc, silver and other metals or mixtures, salts or solutions thereof, and other components. One embodiment of the invention relates to the use of Fe₂(SO₄)₃ or other metal compounds of the general formula Meₓ(SO₄)_{y} in solution applied to organic waste, where Me is a metal and x and/or y are 2 or 3. Preferably, metal compounds to be used for counteracting hydrogen sulfide emissions include but are not limited to copper sulfate, zinc sulfate, ferric sulfate and ferrous sulfate.

Certain embodiments of the invention relate to retaining the nitrogen as ammonium sulfate, for use in future application as fertilizer in agriculture. The use of metal sulfate solutions enables the neutralization of hydrogen sulfide gas while forming H₂SO₄, which is absorbed by the NH₃. The nitrogen is retained for the plants in the soil.

Yet another embodiment of the invention relates to the use of compounds of the general formula Meₓ(NO₃)_{y} in solution applied to organic waste, where Me includes but is not limited to silver, copper, iron and zinc. Preferably, metal compounds to be used for counteracting hydrogen sulfide emissions include but are not limited to silver nitrate.

In another embodiment, zinc acetate Zn(CH₃COO)₂ is used, instead of Meₓ(SO₄)_{y} or Meₓ(NO₃)_{y}, in the form of a solution. When used in the field, the zinc acetate forms acetic acid, which is biodegradable.

The malodor caused by the presence of hydrogen sulfide, ammonium and other compounds can be treated by treating the organic waste, a wastewater source or manure, directly to neutralize the effects of the hydrogen sulfide and/or by reducing or neutralizing the hydrogen sulfide concentration in the air.

It is an object of this invention to provide an improved treatment of organic waste to eliminate the offensive and toxic gases emitted by the waste.

It is a further object of this invention to provide an improved treatment for organic waste to eliminate hydrogen sulfide gas emitted by the waste.

Still other objects and advantages of the invention will in part be obvious and will in part be apparent from the specification.

The invention accordingly comprises the several steps and the relation of, one or more of such steps with respect to each of the others, and the composition of matter embodying features of construction, combinations of elements and arrangements of parts which are adapted to effect such steps, all as exemplified in the following detailed disclosure, and the scope of the invention will be indicated in the claims.

### DETAILED DESCRIPTION OF THE PREFERRED EMBODIMENTS

It has been determined that hydrogen sulfide gas emitted or generated by organic waste can be treated with a mixture of components, such as metal sulfates, nitrates and acetates, to abate its offensive odors and neutralize its toxicity to surrounding communities.

In one embodiment of the invention, hydrogen sulfide gas is abated with the treatment of effective amounts of compounds of the general formulas Meₓ(SO₄)_{y} and/or Meₓ(NO₃)_{y} and/or Meₓ(CH₃COO)_{y} and/or solutions thereof. These compounds can optionally be combined with any or all of an alcohol, a surfactant, a fragrance and a preservative. The surfactants used should have good compatibility with many solvents and excellent biodegradability. The preservatives used should have good compatibility with surfactants and emulsifiers and rapid biodegradability in the environment.

Suitable metals that can be used include but are not limited to copper, iron, zinc, silver and other metals or mixtures, salts or solutions thereof. The amount of metals such as copper, iron, zinc, silver and other metals or mixtures and salts thereof, used in the present invention should be effective to reduce odors by a perceivable level. This is advantageously in the range from about 1 ppm to about 50,000 ppm preferably in the range from about 10 ppm to about 5,000 ppm.

Suitable fragrances optionally used include but are not limited to benzaldehyde, pine and citrus. Other types of fragrances can be used. The amount of fragrance used in the present invention should be effective to improve the odor of the composition. This is advantageously in the range from about 1 ppm to about 50,000 ppm, preferably in the range from about 10 ppm to about 30,000 ppm.

Suitable surfactants that can be used include but are not limited to Avanel® Sodium Sulfonates, Cremophor® Solubizers/Emulsifiers, Dowfax® Anionic Disulfonates, Macol® Alcohol/Phenol Ethoxylates, MAFO CAB® Cocamidopropyl Betaine, Plurafac® Nonionic Linear Alcohols, Surfonic® DDP Series-Dodecylphenyl Ethoxylates, Surfonic® DNP Series-Dinonylphenyl Ethoxylates, Surfonic® E Series, Branched Alcohol Ethoxylates, Surfonic® CO Series, Tergitol® Nonionic, Tomadol® Alcohol Ethoxylates and Triton® Nonionic. Preferably, the surfactant used is Tomadol®. The amount of surfactant used in the present invention should be effective to improve the flow characteristics of the composition. This is advantageously in the range from about 1 ppm to about 60,000 ppm, preferably in the range from about 10 ppm to about 30,000 ppm.

Suitable preservatives that can be used in effective amounts include but are not limited to Kathon™ CG, Kathon™ CG II, Kathon 893, Kathon 925 and Neolone™ 950. Preferably, the preservative is Kathon™ CG because it is effective at low use levels, it has good compatibility with surfactants and emulsifiers, irrespective of their ionic nature and has low toxicity at recommended use levels. The amount of preservatives used in the present invention should be effective to prevent degradation. This is advantageously in the range from about 0 ppm to about 100 ppm, preferably in the range from about 10 ppm to about 50 ppm.

Suitable alcohols that can be used include but are not limited to ethanol, propanol and ethyl alcohol-denatured. Preferably, the alcohol is isopropanol because it is the least toxic and cost-effective. The amount of alcohol should be effective to This is advantageously used in the present invention is typically in the range from about 1 ppm to about 200,000 ppm, preferably in the range from about 10,000 ppm to about 30,000 ppm.

Compositions for abating organic waste emissions can be represented by, but are not limited to, the following examples, which are not intended to be construed as limiting.

### Stoichiometric Calculation

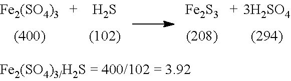

Therefore, for every 1 ppm of H₂S, we need 3.92 ppm Fe₂(SO₄)₃, a ratio of about 1:4.

The following sample compositions and examples are provided for the purpose of illustration only, and are not intended to be controlling or limiting the scope of the invention.

### Sample Composition 1

| Component | Amount |
|---|---|
| Alcohol | 20% |
| Fragrance | 1-2% |
| Surfactant | 1-3% |
| Preservative | 0.005% |
| Water | 74-76% |
| Zinc Sulfate | 0.05% |
| Copper Sulfate | 0.05% |

### Sample Composition 2

| Component | Amount |
|---|---|
| Alcohol | 5-15% |
| Fragrance | 1-2% |
| Surfactant | 1-3% |
| Preservative | 0.002% |
| Water | 90.7% |
| Ferric Sulfate 60% Solution | 0.4% |

### Sample Composition 3

| Component | Amount |
|---|---|
| Alcohol | 5-15% |
| Fragrance | 1.5-3.0% |
| Surfactant | 4-6% |
| Water | 71.0-86.5% |
| Ferric Sulfate 60% Solution | 3.0-5.0% |

### Sample Composition 4

| Component | Amount |
|---|---|
| Alcohol | 5-15% |
| Fragrance | 1-3% |
| Surfactant | 2.5% |
| Preservative | 0.002% |
| Water | 85.8% |
| Ferric Sulfate 60% Solution | 0.2% |

### Example 1

| Component | Amount |
|---|---|
| Isopropyl Alcohol | 10% |
| Fragrance | 1.5% |
| Surfactant | 2.5 |
| Preservative | 0.002% |
| Water | 85.8% |
| Ferric Sulfate 60% Solution | 0.2% |

i. Air Treatment of Hydrogen Sulfide

Using a trigger spray bottle, 6-7 ounces of the composition of Example 1 was sprayed into a room housing a dumpster containing wastewater. The volume of the room was 5586 ft³. The room measured about 45 ppm of hydrogen sulfide in the air. After spraying the room, the concentration of the hydrogen sulfide dropped from about 45 ppm to 0 ppm as measured by a T40 Ratter Instrument. Only after one hour did the concentration begin to slowly increase.

ii. Air Treatment of Hydrogen Sulfide

The composition of Example 1 was prepared and two pails of solution were tested in the field using a Viking pump to continuously spray the product into the air. The solution was diluted with water before use by adding one part water for every one part composition used. The concentration of hydrogen sulfide dropped from about 45 ppm to 0 ppm. When treating the air, the composition should be diluted up to four times.

These formulations are stable at temperatures between about 40° F. to 130° F. making storage and transportation feasible. They can be used at the given concentrations for wastewater treatment or can be diluted for treatment of manure or spraying into the air.

iii. Wastewater Treatment of Hydrogen Sulfide

The composition of Example 1 was added to a sample of wastewater in the amount of 0.5 ml for every 100 ml of liquid. The wastewater contained about 30 ppm hydrogen sulfide. After the composition of Example 1 was added, the amount of hydrogen sulfide was reduced to 10 ppm as measured by a T40 Rattler Instrument.

Hydrogen sulfide can be detected only after water has been saturated. Saturation is a function of temperature. The following table shows the amount of hydrogen sulfide present in a water solution at varying temperatures.

| ° F. | ° C. | Hydrogen Sulfide (ppm) |
|---|---|---|
| 32 | 0 | 4.670 |
| 41 | 5 | 3.970 |
| 50 | 10 | 3.399 |
| 59 | 15 | 2.945 |
| 68 | 20 | 2.582 |
| 77 | 25 | 2.282 |
| 86 | 30 | 2.037 |
| 95 | 35 | 1.831 |
| 104 | 40 | 1.660 |
| 176 | 80 | 0.917 |

It will thus be seen that the objects set forth above, among those made apparent from the preceding description, are efficiently attained and, since certain changes may be made in carrying out the above compositions and methods in using those compositions and in the constructions set forth without departing from the spirit and scope of the'invention, it is intended that all matter contained in the above description and shown in the accompanying drawings shall be interpreted as illustrative and not in a limiting sense.

It is understood to be that the following claims are intended to cover all of the generic and specific features of the invention herein described and all statements of the scope of the invention which, as a matter of language, might be said to fall there between.

Particularly, it is understood that in said claims, compounds recited in the singular are intended to include compatible mixtures of such compounds wherever the sense permits.

What is claimed is:

1. An odor reducing method for abating hydrogen sulfide gas emitted from agricultural or municipal waste sources, comprising: treating the emitted gas with an effective amount of a treatment composition comprising metal sulfates, metal nitrates or metal acetates, their respective salts or solutions and combinations thereof, effective to at least partially eliminate the hydrogen sulfide gas, and thereby reducing said hydrogen sulfide gas.

2. The method of claim 1 wherein the metal is at least one of copper, iron, zinc, or silver.

3. The method of claim 1, wherein the treatment composition comprises copper sulfate.

4. The method of claim 1, wherein the treatment composition comprises iron sulfate.

5. The method of claim 1, wherein the treatment composition comprises zinc acetate.

6. The method of claim 1, wherein the treatment composition comprises silver nitrate.

7. The method of claim 1, wherein the treatment composition is used to reduce hydrogen sulfide emissions from a wastewater treatment facility.

8. The method of claim 1, wherein the treatment composition is used to reduce hydrogen sulfide emissions from an animal breeding farm.

9. The method of claim 1, wherein the composition is added to the waste source in an amount sufficient to achieve a concentration in the range from about 1 ppm to about 50,000 ppm.

10. The method of claim 1, comprising the step of adding a sufficient amount of an alcohol to the waste source to achieve a concentration in the range from about 1 ppm to about 200,000 ppm.

11. The method of claim 1, comprising the step of adding a sufficient amount of a fragrance to a waste source to achieve a concentration in the range from about 1 ppm to about 50,000 ppm.

12. The method of claim 1, comprising the step of adding a sufficient amount of surfactant to a waste source to achieve a concentration in the range from about 1 ppm to about 60,000 ppm.

13. The method of claim 1, wherein the fragrance comprises benzaldehyde, pine, citrus or combinations thereof.

14. An odor treatment composition, formulated to react with and abate hydrogen sulfide gas emitted from waste sources, comprising about 0.1%-5% Meₓ(SO₄)_{y} and/or Meₓ(NO₃)_{y} and/or Meₓ(CH₃COO)_{y}, about 5-20% alcohol, about 1-3% fragrance, about 1-6% surfactant and 0 to about 0.1% preservative, where Me is at least one of copper, iron, zinc or silver, and x and y are appropriate integers.

15. The composition of claim 14, wherein the fragrance comprises benzaldehyde, pine, citrus or combinations thereof.

16. The composition of claim 14, wherein the composition comprises copper sulfate.

17. The composition of claim 14, wherein the composition comprises iron sulfate.

18. The composition of claim 14, wherein the composition comprises zinc acetate.

19. The composition of claim 14, wherein the composition comprises silver nitrate.
